(19) United States
(12) Patent Application Publication
(10) Pub. No.: US 2024/0352392 A1
(43) Pub. Date: Oct. 24, 2024

(54) MACROENCAPSULATION DEVICES

(71) Applicant: Vertex Pharmaceuticals Incorporated, Boston, MA (US)

(72) Inventors: Christopher Thanos, Providence, RI (US); John Mills, Warwick, RI (US); Matthew Watson, Stoneham, MA (US); Rahul R. Rajendran, Shrewsbury, MA (US); Noah Nguyen, Cranston, RI (US)

(73) Assignee: Vertex Pharmaceuticals Incorporated, Boston, MA (US)

(21) Appl. No.: 18/682,738

(22) PCT Filed: Aug. 16, 2022
(86) PCT No.: PCT/US2022/040393

§ 371 Date: Feb. 9, 2024

Related U.S. Application Data
(60) Provisional application No. 63/233,667, filed on Aug. 16, 2021.

(51) Int. Cl. C12M 1/12

(52) U.S. Cl. CPC C12M 25/16; C12M 25/02

(57) ABSTRACT

Macroencapsulation devices and related methods of manufacture are described where bonded membranes of a device may be mounted to an associated frame in an arrangement to provide stress relief between the frame and a seal perimeter of the bonded membranes. The seal perimeter may be arranged radially inward from an outer perimeter of the membranes such that when the membranes are mounted to a corresponding perimeter frame, the seal perimeter is spaced radially inwards from the frame with a unbonded portion of the one or more membranes disposed between the frame and seal perimeter.

### CROSS-REFERENCE TO RELATED APPLICATION

This application claims the benefit under 35 U.S.C. § 119 (e) to U.S. Provisional Application Ser. No. 63/233,667, filed Aug. 16, 2021, which is herein incorporated by reference in its entirety.

### FIELD

Disclosed embodiments are related to macroencapsulation devices and their methods of manufacture.

### BACKGROUND

Therapeutic devices that deliver biological products can be used to treat metabolic disorders, such as diabetes. The therapeutic devices may be implantable to provide a biological product, such as insulin, for an extended period of time. Some of these devices include macroencapsulation devices used to house cells to produce the desired biological product, a matrix including the cells, or other desired therapeutics within.

### SUMMARY

Various embodiments of macroencapsulation devices are described herein. The various embodiments of the disclosed macroencapsulation devices may provide improvements related to the manufacturability, fatigue resistance, compatibility with automation, and/or other benefits as described in further detail below.

In one embodiment, a macroencapsulation device for housing a population of cells comprises a first membrane and a second membrane disposed on the first membrane. The first membrane and the second membrane are bonded together to form a seal extending around an internal volume disposed between the first membrane and the second membrane. The seal is disposed radially inward from an outer perimeter of the first and second membranes. The first membrane and/or the second membrane is semipermeable. The macroencapsulation device further comprises a frame, wherein the first membrane and the second membrane are disposed on the frame, wherein the frame extends along at least a portion of the outer perimeter of the first and second membranes, and wherein the seal is disposed radially inwards from the frame.

In another embodiment, a macroencapsulation device for housing a population of cells comprises a first membrane and a second membrane disposed on the first membrane. The first membrane and the second membrane are bonded together to form a seal extending around an internal volume disposed between the first membrane and the second membrane. The first membrane and/or the second membrane is semipermeable. The macroencapsulation device further comprises a frame disposed on the first membrane or second membrane that extends along at least a portion of the perimeter of the first and second membranes, wherein the frame includes a fill port extending from an exterior portion of the frame to an interior portion of the frame, and wherein an opening of the fill port located on the interior portion of the frame is in fluid communication with the internal volume and is flush with an adjacent portion of the internal portion of the frame.

In another embodiment, a method of forming a macroencapsulation device comprises depositing a first membrane and a second membrane onto a frame, wherein the first membrane and the second membrane are bonded together to form a seal extending around an internal volume disposed between the first membrane and the second membrane, and wherein the seal is disposed radially inward from an outer perimeter of the first and second membranes. The method further comprises connecting the frame to the second membrane and/or the first membrane along the outer perimeter of the first and second membranes at one or more locations located radially outward from the seal.

In yet another embodiment, a method of forming a macroencapsulation device comprises disposing a first membrane and a second membrane on a first surface of a frame, wherein the frame includes a fill port extending from an exterior portion of the frame to an interior portion of the frame, and wherein the frame includes a second surface located opposite from the first surface. The method further comprises disposing a first flap of the first membrane on a portion of the first surface adjacent to the fill port, disposing a second flap of the second membrane on a portion of the second surface adjacent to the fill port such that a portion of the fill port is disposed between the first flap and the second flap, and sealing the first flap and the second flap with the frame such that the fill port is in fluid communication with an interior volume disposed between the first membrane and the second membrane.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1A is a front view of a bonded membrane of a macroencapsulation device prior to mounting to a frame, according to one embodiment;

FIG. 1B is a side view of the embodiment of FIG. 1A;

FIG. 2A is a front perspective view of a frame of a macroencapsulation device, according to one embodiment;

FIG. 2B is an enlarged view of section 2B of the embodiment of FIG. 2A;

FIG. 2C is a front view of the frame of the embodiment of FIG. 2A;

FIG. 2D is a front enlarged view section 2D of FIG. 2C;

FIG. 2E is a side view of the frame of the embodiment of FIG. 2A;

FIG. 2F is an enlarged side view of section 2F of FIG. 2E;

FIG. 3A is a front view of a macroencapsulation device, according to one embodiment;

FIG. 3B is an enlarged perspective view of a section of the embodiment of FIG. 3A;

FIG. 3C is a side view of the macroencapsulation device of the embodiment of FIG. 3A;

FIG. 3D is a side view of the macroencapsulation device of the embodiment of FIG. 3A after loading with a desired material;

FIG. 4 shows a process for connecting a bonded membrane to a frame, according to one embodiment;

FIG. 5A is a front view of a macroencapsulation device exhibiting fatigue failure due to stress concentrations at a bonded membrane perimeter; and

FIG. 5B is a front view of a macroencapsulation device exhibiting fatigue failure due to stress concentrations at an elongated fill port.

### DETAILED DESCRIPTION

Driven by a rising need to deliver biological products to treat metabolic disorders, such as diabetes, different types of implantable therapeutic devices have been engineered. However, the Inventors have recognized that typical methods of making such devices are often cumbersome and hard to control. For instance, there is often a lack of precision and control in forming specific structural features (e.g., mounting adhesive application) associated with the device. In addition, the Inventors have recognized that it is oftentimes difficult to precisely form these devices within accepted tolerances to prevent mechanical failure of these devices once implanted.

For example, in some embodiments, during a manufacturing process of a macroencapsulation device, at least one, and in some instances two or more flexible membranes of the device may be bonded together to form a seal perimeter extending around an internal volume disposed between the membranes. The flexible bonded membrane may be mounted to a corresponding semi-rigid frame and the two may be bonded together. The Inventors have recognized that the transition from the semi-rigid frame to the flexible membrane may lead to high stress concentrations at the membrane frame interface. Additionally, imperfections in the adhesive application at this interface may increase the localized stress on the membrane during repeated flexing of the device when implanted. These stress concentrations may result in delamination and/or membrane rupture due to fatigue failure. Other structural features of the frame, such as a fill port that extends into the internal volume disposed between the membranes, may also increase the local stresses applied to the membranes which may again lead to accelerate fatigue failure and membrane rupture during use.

In view of the above, the Inventors have recognized the benefits associated with macroencapsulation devices where the relative arrangement of the membranes and frame of the device, as well as adhesive application techniques to bond them together, may be controlled to modify one or more parameters of the resulting macroencapsulation devices. For example, a relative sizing and placement of the membranes and the associated frame may provide a simple and easily controllable method for producing macroencapsulation devices with low stress and risk of failure of the membranes at the frame interface. This may include providing stress relief between a frame and the seal perimeter of the bonded membranes held in the frame to permit the flexible unbonded membrane to accommodate relative deformation between the more rigid frame and seal perimeter. For example, the seal perimeter may be arranged radially inward from an outer perimeter of the membrane such that when the membrane is mounted to a corresponding perimeter frame, the seal perimeter is spaced radially inwards from the frame with a unbonded portion of the one or more membranes disposed between the frame and seal perimeter. The space between the frame and the seal perimeter may create a stress buffer, which may also be referred to as a buffer region herein, between the frame and the membrane seal perimeter which may reduce fatigue failure of the membrane.

The Inventors have further recognized that it may be desirable to prevent spread of an adhesive used to bond one or more membranes of a device to an associated frame into undesirable adjacent locations of the membrane. This may include limiting the spread of adhesive into a buffer region between a frame and a seal perimeter extending at least partially around an internal volume of a device. The technique may include initial bonding locations arranged around the perimeter of the frame. The bonding sites may include reservoirs in the frame perimeter into which liquid adhesive is deposited and allowed to wick therefrom into the surrounding portions of the membranes. The viscosity, adhesive amount, reservoir size, and membrane properties may be selected to permit the adhesive to cure and bond the membrane to the frame while limiting the spread of the adhesive to the desired locations. After the membrane is bonded to the frame at each bonding site, a second adhesive application may be used to deposit either the same, or a different adhesive, around the frame perimeter in sections between and/or around the bonding sites to create a strong bond between the membranes and the frame. In some embodiments, both adhesive applications may leave an unbonded portion of the membranes disposed between the frame and a seal perimeter of the membranes to provide the above noted stress buffer.

Depending on the particular embodiment, the reservoir formed in a frame for receiving an adhesive during manufacture may have any appropriate size and/or shape. For example, in some embodiments, a size of the reservoirs included in a frame may be between about 50 μL and about 500 μL. In some embodiments, a size of the reservoirs included in a frame has an average volume of about 250 μL. In some embodiments, a size of the reservoir included in a frame is scaled by volume depending on design. For example, in some embodiments, a size of the reservoir included in a frame is about 1.6 L/cm. Additionally, depending on the specific embodiment, the reservoirs may cover a surface area of a mounting surface on which the membranes are disposed by any desired amount including, for example, greater than or equal to 10%, 25%, and/or 50% of the surface area of the portion of the frame the membranes are mounted to it. Correspondingly, the reservoirs may cover less than or equal to 80%, 75%, 50%, and/or 25% of the surface area of the portion of the frame the membranes are mounted to. Combinations of the foregoing ranges are contemplated including, for example, the reservoirs may cover between or equal to 10% and 80% of the surface area of a portion of the frame the membranes are mounted to. Individual reservoir volumes and areal coverage both greater than and less than those noted above are also contemplated as the disclosure is not so limited.

In addition to the above, the Inventors have recognized that it may be desirable to avoid applying stresses to the membranes of a device due to the inclusion of structures extending into an interior volume of the device disposed between opposing portions of the one or more membranes. Thus, in some embodiments, an opening of a fill port in fluid communication with the interior volume may be flush with an adjacent inner portion of the frame (i.e., the fill port does not extend into the interior volume formed by the one or more membranes). This may reduce or eliminate stress concentrations and potential stresses applied to the membranes due to the use of a fill port extending into the membrane. The Inventors have recognized and appreciated techniques for sealing the bonded membrane around the flush opening which are expanded on further below. A population of cells has been shown to flow into a sealed interior volume of a bonded membrane with a flush-mounted opening just as well as with a fill port that extends into the membrane. For example, during testing of devices with flush fill ports and fill port extensions, the measured filling efficiency of devices with a flush fill port were 93.33% filled whereas devices with fill port extensions were 90% filled. In embodiments including the flush fill ports, the measured filling efficiency may be equal to or greater than approximately 85%, 90%, 91%, 92%, 93, 94%, and/or 95%. The filling efficiency may also be less than or equal to approximately 99.99%, 99%, 98%, 97%, 96%, and/or 95%. In embodiments including the fill port extensions the measured filling efficiency may be between or equal to approximately 80% and 99.99%, or more preferably between or equal to 90% and 99.99%. However, other combinations of the above ranges may also be used.

As noted above, a macroencapsulation device may include multiple layers of membranes. At least one exterior membrane of these multiple layers of membranes may be semipermeable. However, embodiments in which each of the membranes is semipermeable or where at least one of the membranes within a device are substantially impermeable are also contemplated. Further, a device may include two stacked membranes, three stacked membranes, and/or any other appropriate number of membranes as the disclosure is not limited in this fashion. For example, in one embodiment including two membranes, either membrane may be semipermeable and the other impermeable or both may be semipermeable. Accordingly, it should be understood that the current disclosure is not limited to any particular combination of membranes within a stacked structure.

In some embodiments, a macroencapsulation device may include at least one population of cells disposed within an internal volume of the device. For example, the population of cells may be disposed within an internal volume formed between two or more opposing exterior membranes of the device where an exterior edge of the internal volume may be defined by one or more bonds extended around at least a portion, and in some instances an entire, perimeter of the membranes or other appropriate portion of the membranes. In such an embodiment, at least the exterior membranes of the device may be configured to block passage of the one or more populations of cells out of the device. Accordingly, the one or more populations of cells may be retained within the interior volume of the device. While the use of two exterior membranes forming a single internal volume is noted, the use of multiple intermediate membranes positioned between the exterior membranes of a device and/or multiple unconnected interior volumes within a device are also contemplated. Additionally, instances in which a single membrane is folded over and bonded to itself to provide two opposing membranes to form the interior volume are also contemplated.

In addition to retaining a population of cells within an interior of a device, in some embodiments, the membranes of a device may be configured to protect the one or more populations of cells disposed in an interior of the device from an immune attack while permitting the passage of a desired biological product, such as insulin, produced by the cells as well as waste and nutrients used and produced by the cells. In some embodiments, the membranes are configured to protect the cells from an immune attack in the absence of an immune suppression therapy. Depending on the particular embodiment, the desired exchange properties and immune response protection properties of the membrane may be based on: size exclusion where a pore size distribution of the membrane is selected to exclude immune cells based on size; balancing the diffusion kinetics of the larger immune cells through the membranes such that it is significantly less than the diffusion of the desired biological product, cell waste, and nutrients through the use of pore size, tortuosity, membrane thickness, and other appropriate parameters; combinations of the foregoing; and/or other appropriate exclusion techniques.

The membranes of a macroencapsulation device may be formed from any appropriate biocompatible material. The biocompatible material may be substantially inert towards cells housed within the macroencapsulation device and the surrounding tissue. The biocompatible material may comprise a synthetic polymer or a naturally occurring polymer. In some embodiments, the polymer may also be a linear polymer, a cross linked polymer, a network polymer, an addition polymer, a condensation polymer, an elastomer, a fibrous polymer, a thermoplastic polymer, a non-degradable polymer, combinations of the foregoing, and/or any other appropriate type of polymer as the disclosure is not limited in this fashion. In one embodiment, a polymer may comprise expanded polytetrafluoroethylene (cPTFE). Appropriate types of polymers may also comprise polyvinylchloride (PVC), polyethylene (PE), polypropylene (PP), polymethylmethacrylate (PMMA), polystyrene (PS), polytetrafluoroethylene (PTFE), expanded polytetrafluoroethylene (ePTFE), polyurethane (PU), polyamide (nylon), polyethyleneterephthalate (PET), polyethersulfone (PES), polyetherimide (PEI), polyvinylidene difluoride (PVDF), polycaprolactone (PCL), poly (lactic-co-glycolic acid) (PLGA), poly-L-lactide (PLLA), polyacrylonitrile (PAN), electrospun PAN/PVC, any combination of the foregoing, and/or any other appropriate polymeric material. In some embodiments, a membrane used with any of the embodiments disclosed herein may comprise PVDF. In some embodiments, a membrane used with any of the embodiments disclosed herein may comprise electrospun PAN PVC. In some embodiments, a membrane used with any of the embodiments disclosed herein may comprise PES. In some embodiments, a membrane used with any of the embodiments disclosed herein may comprise PS. In some embodiments, a membrane used with any of the embodiments disclosed herein may comprise PAN. In some embodiments, a membrane used with any of the embodiments disclosed herein may comprise polycarbonate. In some embodiments, a membrane used with any of the embodiments disclosed herein may comprise polypropylene. The synthesis methods used for forming one or more of the porous membranes from the above noted polymeric materials may include, but are not limited to. expansion, solvent-casting, immersion precipitation and phase separation, electrospinning, methods that yield isoreticular network, methods that yield trabecular network, or any other appropriate method of forming a porous polymer membrane.

Sintering of a membrane may be used to alter the porosity and flux properties of a membrane. For example, the sintering may increase the porosity of the membrane while maintaining its pore structure. The sintering may also improve the mechanical stability and diffusive flux of the membrane. Thus, sintering may be used to alter the porosity and/or mechanical properties of the membranes, which in turn can be used to tune the porosity and the flux properties of the macroencapsulation device. Accordingly, in some embodiments, any desired combination of sintered and/or unsintered membranes may be used. For instance, two exterior membranes of a device may be bonded together where either a sintered and unsintered membrane are bonded together, two sintered membranes are bonded together, or two unsintered membranes are bonded together. Further, any number of intermediate membranes positioned between these exterior membranes may be used where these intermediate membranes may be sintered or unsintered.

The membranes of a macroencapsulation device as described herein may be made from porous membrane materials that are configured to allow for transport through the membranes of materials, such as a biological product, with a molecular weight less than about 3000 kDa, 2000 kDa, 1000 kDa, 500 kDa, 400 kDa, 300 kDa, 200 kDa, 100 kDa, 50 kDa, 40 kDa. 30 kDa, 20 kDa, 10 kDa, 6 kDa, 5 kDa, 4 kDa, 3 kDa, 2 kDa, 1 kDa, and/or any other appropriate range of molecular weights depending on the desired application. For example, the one or more membranes of a macroencapsulation device may be configured to permit the flow of insulin through the membranes which has a molecular weight of about 5.8 kDa.

To provide the desired selectivity, the porous membranes used with the macroencapsulation devices disclosed herein may have an open porous structure with average pore sizes that are greater than or equal to about 1 nm, 5 nm, 10 nm, 15 nm, 20 nm, 30 nm, 40 nm, 50 nm, 60nm, 70 nm, 80 nm, 90 nm, 100 nm, 200 nm, 300 nm, and/or any other appropriate size range. Correspondingly, the average pore size of the various membranes described herein may have an average pore size that is less than or equal to 2500 nm, 2000 nm, 1700 nm, 1500 nm, 1400 nm, 1300 nm, 1200 nm, 1100 nm, 1000 nm, 900 nm, 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20 nm, and/or any other appropriate size range. Combinations of the foregoing are contemplated including, for example, an average pore size that is between or equal to 1 nm and 20 nm, 1 nm and 2500 nm, and/or any other appropriate combination. While specific average pore sizes arc described above, it should be understood that any appropriate average pore size may be used for the various membranes described herein including average pore sizes both greater than and less than those noted above.

In some embodiments, charge exclusion properties may be included in the membrane. For example, a surface charge of the membranes may be modulated with external coatings, plasma treatments, or other surface treatments to achieve neutral, positive, negative, or zwitterionic properties based on the isoelectric point of the desired ancillary agent. The agent may be a protein, complexed small molecule, and/or any other appropriate agent depending on the desired application.

To provide sufficient strength and/or rigidity for a macroencapsulation device, the various membranes and frames may be made from materials that are sufficiently stiff. The desired stiffness may be provided via an appropriate combination of a material's Young's modulus (also referred to as an Elastic modulus), thickness, and overall construction which may be balanced with a desired permeability of the device. Appropriate Young's moduli for the various membranes and frames described herein may be at least 10⁵ Pa, 10⁶ Pa, 10⁷ Pa, 10⁸ Pa, 10⁹ Pa, and/or 10¹⁰ Pa. Other appropriate Young's moduli for the various membranes and frames described herein may be used including moduli both greater than and less than these ranges. Ranges between the foregoing Young's moduli are contemplated including, for example, a Young's modulus between or equal to about 10⁶ Pa and 10¹⁰ Pa. In some embodiments, an appropriate material for the frame may include polyetheretherketone (PEEK). Appropriate materials for the frame may also include, but are not limited to polycarbonate, polyurethane, polyetheretherketone (PEEK), Polyvinyl Chloride (PVC), poly (oxymethylene), poly (methyl methacrylate) (PMMA), thermoplastic polymer based composites, polypropylene, fluorinated ethylene propylene (FEP), low density polyethylene (LDPE), high density polyethylene (HDPE), ultra-high density polyethylene (UHDPE), polycaprolactone, poly (lactide), poly (glycolic acid), poly lactide-co-glycolide, ethylene vinyl acetate copolymer, polyamides, poly (butylene) therephthalate, and combinations of the forgoing. In some embodiments, an appropriate material for the frame includes polyetheretherketone (PEEK). In some embodiments, an appropriate material for the frame includes polypropylene. In some embodiments, an appropriate material for the frame includes fluorinated ethylene propylene (FEP). In some embodiments, an appropriate material for the frame includes ultra-high density polyethylene (UHDPE). In other embodiments. an appropriate material for the frame or portion of the frame may include titanium, graphene, stainless steel, or other appropriate biocompatible material exhibiting sufficient rigidity to function as a frame for the macroencapsulation device.

In some embodiments, it may be desirable for one or more of the membranes included within a macroencapsulation device to be hydrophilic to facilitate loading of cells into the macroencapsulation device and/or to facilitate the flow of one or more fluids, biological compounds, therapeutics, cell nutrients, cell waste, and/or other materials through the membranes of a device. Additionally, a hydrophilic outer membrane may also reduce the occurrence of fibrosis when the device is positioned in vivo. Accordingly, the membranes of a macroencapsulation device may either be made from a hydrophilic material and/or treated with a hydrophilic coating. Appropriate hydrophilic coatings may include, but are not limited to polyhydroxyacrylate, PEG, pHPA, carboxymethylcellulose, alginate, agarose, and/or solute-impregnated thermoplastic coatings. Appropriate hydrophilic materials may also include, but are not limited to an appropriate hydrophilic polymer, polyethylene glycol, polyvinyl alcohol, polydopanine, any combination thereof, and/or any other appropriate hydrophilic material capable of forming a coating on the membranes or that the membranes may be made from.

The membranes described in the various embodiments of macroencapsulation devices described herein may be bonded to one another using any appropriate bonding method as the disclosure is not limited in this fashion. For example, adjacent membranes may be bonded to one another using an adhesive, an epoxy, a weld or other fusion based technique (e.g. ultrasonic bonding, laser bonding, physical bonding, thermal bonding, etc.), mechanical clamping using a frame or fixture, and/or any other appropriate bonding method. In one specific embodiment, adjacent membranes may be bonded using a heated tool that is used to press or strike two or more membranes against each other for a set fusion time with a predetermined pressure and/or force. In view of the above, it should be understood that the current disclosure is not limited to the use of any particular method for bonding the membranes together.

In some embodiments, one or more thermal treatments may be applied to a stack of bonded membranes after the membranes have been bonded to each other, and in some instances after a frame has been attached to the membranes. For example, the membranes may be bonded together with a bond extending along a perimeter of the membranes and/or one or more bonds may be formed within an interior area of the membranes (e.g. within the bonded perimeter) prior to heat treatment of the membranes. This post bonding heat treatment may provide enhanced bonding of the membranes at the bonded regions. The specific heat treatment temperatures and durations to improve the bonding between the membranes may vary depending on the specific materials used. However, in some embodiments the heat treatment temperature may be between a glass transition temperature and a melting temperature of a polymer membrane.

In certain embodiments, it may be desirable to limit a maximum thickness of a macroencapsulation device in a direction perpendicular to a plane in which a maximum transverse dimension of the device lies. Accordingly, one or more interior portions of first and second membranes disposed within a frame may be bonded together to limit the extent to which the membranes may be displaced relative to one another. These bonded portions of the membranes may be dispersed uniformly within the interior portion of the membranes located within the frame. These bonded portions may have any appropriate shape including, for example, dots, lines, curves, or any other appropriate shape. While the bonded interior portions may have any appropriate size for a desired application, in one embodiment using bonded dots, the diameter of the bonded dots may be greater than or equal to about 0.5 mm, 0.75 mm, 1 mm, 1.25 mm, 1.5 mm, and/or any other appropriate diameter. Correspondingly, the diameter of the dots may be less than or equal to about 3 mm, 2.75 mm, 2.5 mm, 2.25 mm, 2.0 mm, and/or any other appropriate diameter. Combinations of the above noted ranges are contemplated including, for example, a diameter that is between or equal to 0.5 mm and 3 mm. While specific shapes and size ranges are provided above, it should be understood that other shapes and sizes both smaller and greater than those noted above are contemplated as the disclosure is not limited in this fashion.

In some embodiments, it may be desirable to improve the vascularization of a macroencapsulation device. Accordingly, in certain embodiments, one or more through holes may be formed in the one or more bonded portions located within an interior portion of the membranes disposed radially inwards from a frame of the device. These through holes may permit vasculature to growth through the through holes in addition to growing around the upper and lower surfaces of the device. The one or more through holes may be formed in the bonded portions of the membranes using laser ablation, mechanical puncture, cutting, or any other appropriate method of forming a through hole in the one or more bonded portions of the membranes. As described in further detail herein, in some embodiments, the one or more through holes may also be disposed radially inward relative to both an unbonded stress buffer region and a seal perimeter extending around a perimeter of a sealed internal volume of the device. This may help to avoid the formation of stress concentrations within the membranes adjacent to the frame.

Depending on the particular size of the bonded portions of a membrane, different size through holes may be used. For example, in some embodiments, a through hole formed in a bonded portion of a membrane may have a maximum transverse dimension, such as a diameter, that is greater than or equal to 0.25 mm, 0.5 mm, 0.75 mm, 1.0 mm, 1.25 mm, 1.5 mm, and/or any other appropriate maximum transverse dimension. Correspondingly, the maximum transverse dimension of the through hole may be less than or equal to 2.0 mm, 1.5 mm, 1.25 mm, 1.0 mm, 0.75 mm, 0.5 mm, and/or any other appropriate maximum transverse dimension. Combinations of the above-noted ranges are contemplated including, for example, a maximum transverse dimension of the through holes formed in corresponding bonded portions of a membrane may be between or equal to 0.25 mm and 2.0 mm where the maximum transverse dimension of the through holes is also less than a corresponding maximum transverse dimension of the bonded portions of the membrane they are formed in. While particular dimensions are noted above, it should be understood that other ranges both greater than and less than those noted above are also contemplated as the disclosure is not so limited.

In some embodiments, the above noted bonded portions within an interior area of the device, and the corresponding through holes, may be formed prior to mounting a frame on the device while the membranes are located in a flat planar configuration. This may simplify the manufacturing process when dealing with flexible membranes mounted to a frame with a desired amount of slack which may complicate forming other features after being mounted to the frame.

As elaborated on below, in some embodiments, one or more portions of adjacent membranes may be bonded together such that the interior volume within the device is subdivided into a plurality of interconnected channels, which in some embodiments may be shaped like a lumen though any appropriate shape or configuration of the channels may also be used. The channels may have an inner maximum transverse dimension, such as an inner diameter, that is greater than or equal to 40 μm, 50 μm, 100 μm, 200 μm, 300 μm, and/or 400 μm. Correspondingly, the channels may have an inner maximum transverse dimension that is less than or equal to 800 μm, 700 μm, 600 μm, 500 μm, and/or 400 m. Combinations of the foregoing are contemplated including, for example, an inner maximum transverse dimension of the plurality of channels that is between or equal to 40 μm and 800 μm. Further, a density of the interconnected channels forming the various compartments of a device may have a density per unity area within a transverse plane of the device that is greater than or equal to about 10 channels/cm², 15 channels/cm², 20 channels/cm², 25 channels/cm², 30 channels/cm², 35 channels/cm², 40 channels/cm², 45 channels/cm², 50 channels/cm², 60 channels/cm², 70 channels/cm², 80 channels/cm², 90 channels/cm², 100 channels/cm², 110 channels/cm², 120 channels/cm², 130 channels/cm², 140 channels/cm², 150 channels/cm², 175 channels/cm², or 200 channels/cm². Ranges extending between any of the above noted density of channels are also contemplated including, for example, a density of channels that is between or equal to about 10 channels/cm² and 200 channels/cm². Though densities both greater than and less than the ranges described above are also contemplated.

A macroencapsulation device as described herein may have any appropriate combination of internal volumes, external dimensions, and/or other appropriate physical parameters. For example, an internal volume encompassed by the outer membranes of a macroencapsulation device may be between or equal to 40 μL and 250 μL. A width, or maximum transverse dimension, of the macroencapsulation device may also be between about 20 mm and 80 mm. Additionally, to provide a desired diffusion of oxygen into the interior of a macroencapsulation device to support cells contained therein, a maximum oxygen diffusion distance from an exterior of the device to an interior portion of the device including a population of cells may be less than 50 μm, 100 μm, 150 μm, 200 μm, 250 μm, 300 μm, 350 μm, 400 μm, 450 μm, or 500 μm. In some embodiments, the maximum oxygen diffusion distance from an exterior of the device to an interior portion of the device including a population of cells is less than or equal to 150 μm. In some embodiments, the maximum oxygen diffusion distance from an exterior of the device to an interior portion of the device including a population of cells is less than or equal to 200 μm. In some embodiments, the maximum oxygen diffusion distance from an exterior of the device to an interior portion of the device including a population of cells is less than or equal to 250 μm. Correspondingly, a maximum thickness, or dimension perpendicular to a maximum transverse dimension, of the overall device and/or an internal volume located within the device may be less than 50 μm, 100 μm, 150 μm, 200 μm, 250 μm, 300 μm, 350 μm, 400 μm, 450 μm, or 500 μm. In some embodiments, the maximum thickness, or dimension perpendicular to a maximum transverse dimension, of the overall device and/or an internal volume located within the device is less than or equal to 500 μm. Further, in some embodiments, an outer surface area to volume ratio of the device may be greater than or equal to about 20 cm⁻¹. 40 cm¹, 60 cm¹, 80 cm¹, 100 cm¹, 120 cm¹, or 150 cm⁻¹. Ranges extending between any of the forgoing values for the various dimensions and parameters as well as ranges both greater than and less those noted above are also contemplated.

While specific dimensions, parameters, and relationships related to the macroencapsulation device and the materials it is made from are described above, it should be understood that dimensions, parameters, and relationships both greater than and less than those noted above are contemplated as the disclosure is not limited in this fashion. Accordingly, any appropriate combination of size, construction, material properties, and/or relative performance parameters may be used for a device depending on the desired application.

In some embodiments, a cell population contained within an interior volume of a macroencapsulation device is an insulin secreting cell population. In some embodiments, the cell population comprises at least one cell derived from a stem cell derived cell. In some embodiments, at least one cell is a genetically modified cell. In some cases, at least one cell is genetically engineered to reduce an immune response in a subject upon implantation of the device, as compared to comparable cells that are not genetically engineered. In some embodiments, the cell population is a stem cell derived cell that is capable of glucose-stimulated insulin secretion (GSIS). For example, an appropriate population of cells may comprise pancreatic progenitor cells, endocrine cells, beta cells, a matrix including one or more of the foregoing, or any combination thereof. Further, a matrix may comprise isolated islet cells, isolated cells from pancreas, isolated cells from a tissue, stem cells, stem cell-derived cells, induced pluripotent cells, differentiated cells, transformed cells, or expression systems, which can synthesize one or more biological products. Optionally, in some embodiments, the matrix may comprise a second type of cells that support the first type of cells that synthesize one or more biological products. In some embodiments, the cells may be encapsulated before being placed within the matrix. In such an embodiment, the cells may be encapsulated in a microcapsule or may be conformally coated. However, naked, i.e., uncoated, cells may also be used.

Depending on the particular embodiment, a therapeutically effective density of cells may be loaded into the interior volume of a macroencapsulation device. Appropriate cell densities disposed within an interior volume may be greater than or equal to about 1000 cells/μL. 10,000 cells/μL, 50,000 cells/μL, 100,000 cells/μL, and/or 500,000 cells/μL. Appropriate cell densities disposed within the compartment may also be less than or equal to about 1,000,000 cells/μL, 500,000 cells/μL, 100,000 cells/μL, 50,000 cells/μL, and/or 10,000 cells/μL. Combinations of the foregoing are contemplated including cell densities between about 1000 cells/μL and 1,000,000 cells/μL. In some embodiments, the cell density is between about 100,000 cells/μL and 1,000,000 cells/μL. Cell densities both greater than and less than those noted above may also be used depending on the desired application and cell types being used.

Depending on the specific application and desired duration of use, a macroencapsulation device may be configured to have any appropriate fatigue life when implanted in vivo within a subject. For example, in some embodiments, a macroencapsulation device may be configured for implantation within the abdominal tissue of a subject where it may be subjected to abdominal contractions during use. Accordingly, in some embodiments, a fatigue life of a macroencapsulation device may be greater than or equal to 50,000 cycles, 60,000 cycles, 70,000 cycles, 80,000 cycles, 90,000 cycles, 100,000 cycles, 150,000 cycles, 200,000 cycles, 300,000 cycles, 400,000 cycles, and/or 500,000 cycles. The fatigue life may also be less than or equal to 200,000 cycles. 100,000 cycles, and/or 80,000 cycles. Combinations of the foregoing ranges are contemplated including, for example, a fatigue life that is between or equal to 50,000 cycles and 200,000 cycles. In some embodiments, the fatigue life is between 1,000 and 50,000 cycles. In some embodiments, the fatigue life is between 50,000 and 100,000 cycles. In some embodiments, the fatigue life is between 100,000 and 500,000 cycles. Devices with a fatigue life both greater than and less than those noted above are also contemplated as the disclosure is not limited in this fashion. For the purposes of this application, a fatigue life of a macroencapsulation device may be determined using the fatigue cycle testing procedures discussed in the example section using a cyclic load of between 12 N and 45 N which is similar to the forces that may be experienced by a device when implanted in vivo within the abdominal tissue of a subject.

The macroencapsulation devices described herein may be implanted in a subject in vivo at various sites. In one example, a device may be implanted in a subject by properitoneal or retrorectus implantation. In other examples, the device can be placed by intra-omental implantation. In another example, the device can be placed by subcutaneous implantation. In another example, the device can be placed by suprahepatic implantation. In some instances, the macroencapsulation devices described herein may be fixed in vivo at an implantation site using any appropriate fixation method including, for example, the application of a tissue adhesive. Appropriate tissue adhesives may include, but are not limited to, fibrin, cyanoacrylate, polyethylene glycol, albumin-based adhesive, polymer-based adhesive, and/or any other appropriate adhesive. In another example, the device may be fixed using platelet-rich plasma and/or any other appropriate fixation method as the disclosure is not limited in this fashion.

During use, a macroencapsulation device may be implanted at any desired location within a subject's body as noted above. When implanted, the macroencapsulation device may be exposed to the environment within the surrounding portion of the subject's body. The population of cells disposed within the macroencapsulation device may produce one or more desired biological compounds that may diffuse out of the macroencapsulation device through the one or more semipermeable membranes of the device. In some embodiments, the one or more biological compounds produced by the cells may treat one or more conditions of the subject. Waste excreted by the population of cells may also diffuse out of an interior volume of the device to the surrounding environment through the one or more semipermeable membranes. Correspondingly, oxygen and nutrients from the surrounding environment may diffuse through the one or more semipermeable membranes to the interior volume of the device to maintain an appropriate environment within the interior volume to support the population of cells. In some embodiments, the one or more semipermeable membranes of the device may also exclude immune cells of the subject from the interior volume of the device as detailed further herein.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein. For the sake of clarity, the figures are described in relation to methods and devices including just a first and second outer membrane bonded to one another. However, it should be understood that the methods and devices described in relation to the figures may include any number of intermediate membranes disposed between these outer membranes as the disclosure is not limited in this fashion.

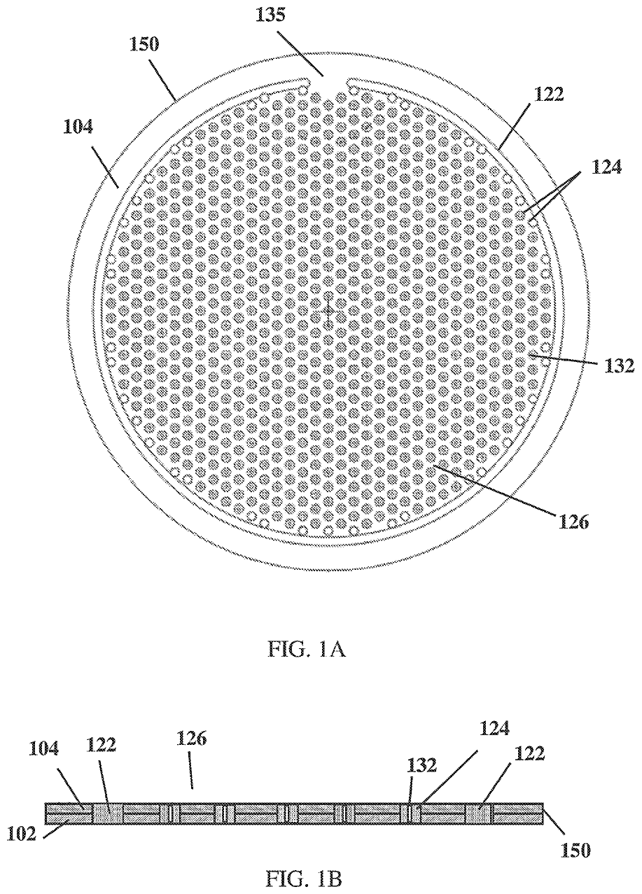

FIGS. 1A-1B show an embodiment of a bonded membrane of a macroencapsulation device prior to mounting to a frame. As shown in the figures, a first and second membrane 102 and 104 may be bonded together at a bonded perimeter 122 and bonded portions 124 located within the bonded perimeter. In FIG. 1A, a top surface of the second membrane 104 is shown with a bonded perimeter 122 of the membranes (e.g., where first and the second membranes are bonded) extending around a perimeter of the bonded membranes. The bonded perimeter 122 may form an interior volume disposed between the first and second membranes. In some embodiments, the bonded perimeter 122 may extend entirely around the perimeter of the membranes; however, as shown in FIG. 1A, the bonded perimeter may have an unbonded portion 135. As will be discussed below, when the membranes are connected to a frame, the unbonded portion 135 may be positioned and sealed around a fill port of the frame such that the fill port is in fluid communication with the interior volume.

As shown in the figures, the bonded perimeter may be disposed radially inward from the outer perimeter 150 of the membranes. The bonded portions 124 may take the form of bonded dots distributed across a surface area of the membranes in a hexagonal array. However, any appropriate shape, arrangement, and/or configuration of these bonded regions may also be used. Due to the presence of these bonded regions located radially inwards from a bonded perimeter of the membranes, an internal volume formed between the membranes, once in the filled configuration, may take the form of a plurality of interconnected channels 126 corresponding to the unbonded regions of the membranes extending between these bonded portions.

In some instances, the bonded portions of the membranes 102 and 104 may have a substantially lower membrane permeability due to the bonding process such that they may be considered non-diffusive portions of the membranes. This may include both the bonded perimeter 122 and the interior bonded portions 124 of the membranes located radially inwards from the bonded perimeter. In contrast, the unbonded portions of the membranes, such as the channels 126 in the depicted embodiment, may be considered diffusive portions of the membranes where the permeabilities of the membranes may be significantly higher than the non-diffusive portions, and in some embodiments may be substantially unaltered from the parent membrane materials. In addition to the bonded portions of the membranes being considered non-diffusive portions of the membranes, portions of the membranes located radially outward from the bonded perimeter 122, and that would not be in direct fluid communication with the resulting interior volume formed there between, may also be considered a non-diffusive portion of the membranes for purposes of this description.

In some embodiments, after bonding portions of the first and second membranes 102 and 104 together, one or more through holes 132 may be formed in one or more of the bonded portions 122 and 124. For example, a device such as a laser, punch, cutter, or other appropriate device may be used to form through holes 132 in one or more of the bonded portions of the first membrane 102 and the second membrane 104. In one specific embodiment, the through holes may be formed via laser ablation where the laser removes a bonded portion of the first and the second membranes while leaving a surrounding bonded portion of the membranes to function as a seal between an interior volume formed by the membranes and an exterior of the device.

As shown in FIG. 1A, some bonded portions 124 located within a specified distance of the bonded perimeter 122 may not include through hoes 132 such that the through holes are disposed radially inward from the bonded perimeter. When the membranes are bonded to a frame, the through holes 132 located near the bonded perimeter 122 may cause stress concentrations when the device is implanted in vivo, resulting in tearing of the membrane at the perimeter 122. Accordingly, while specific dimensions may vary based on the particular design, in some embodiments bonded portions located within less than or equal to approximately 2 mm, 1.75 mm, 1.5 mm, 1.45 mm, or 1.25 mm of the bonded perimeter 122 may not include through holes 132. However, embodiments in which different distances both greater than and less than those noted above are used are also contemplated.

In some embodiments, after bonding the membranes together (e.g. bonding of the perimeter and/or interior portions of the first membrane and the second), the first membrane and the second membrane may be coated with a hydrophilic material and/or subjected to other treatments which may not be compatible with the bonding process. This may include various high temperature treatments where the bonded membranes may be subjected to various thermal treatments which may enhance the bonding of the membranes in some embodiments.

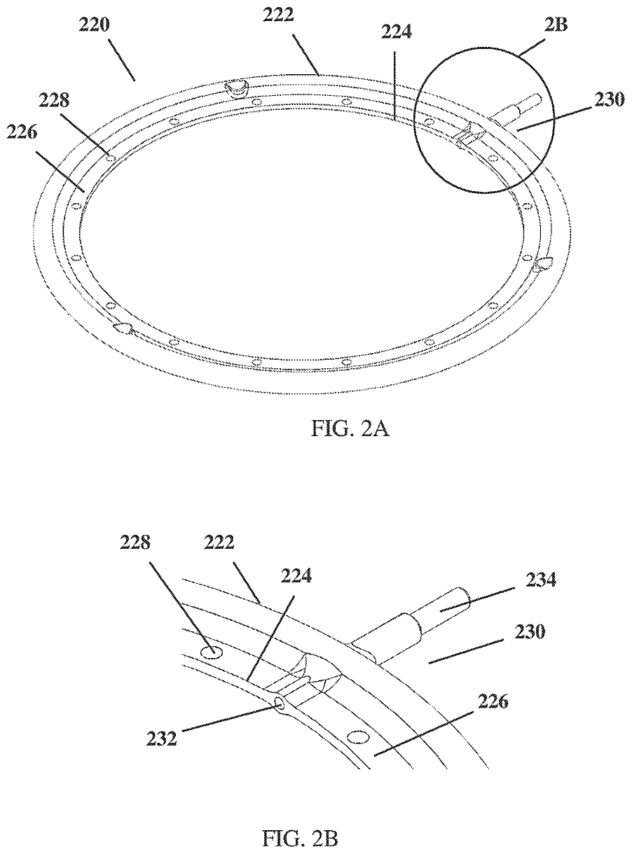

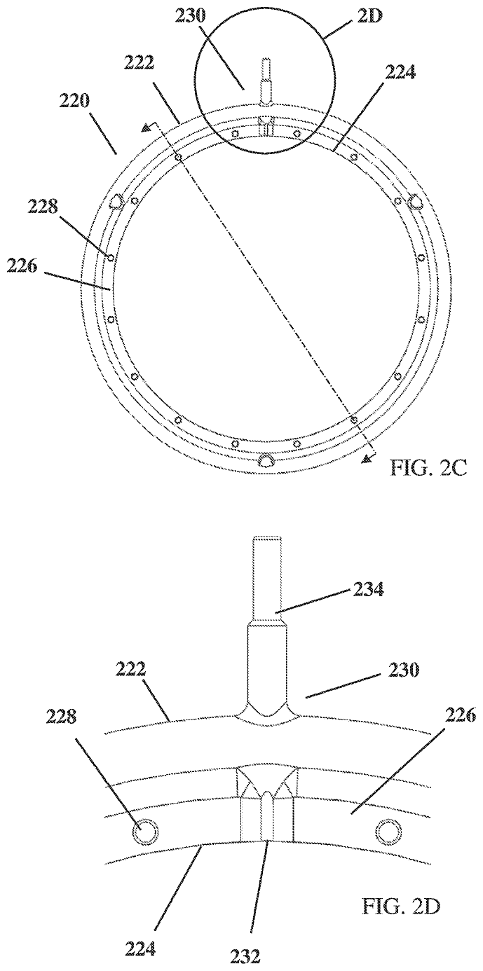

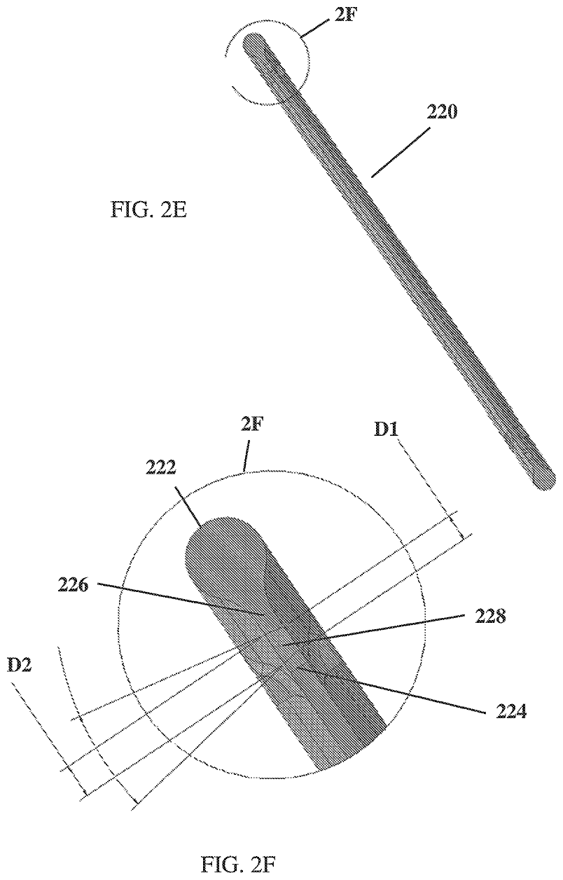

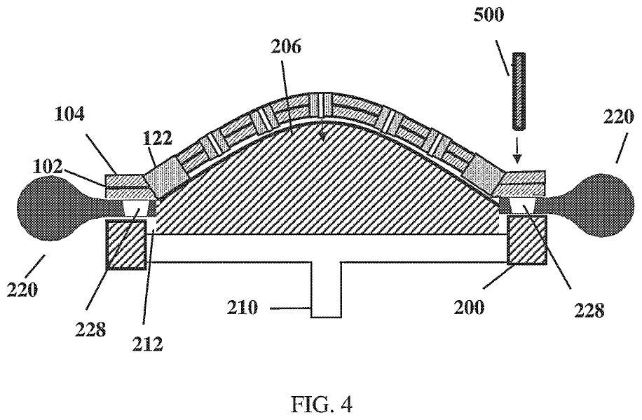

In some embodiments, a prebonded stack of membranes, such as the bonded first and second membranes described above, may be mounted to a frame (see FIGS. 2A-2F). Alternatively, in some embodiments, a perimeter of a stack of membranes may be bonded together and attached to a frame at the same time. In either case, a method for mounting the membranes to a frame to provide a desired amount of slack in the membranes once mounted may be used. One such embodiment is described in further detail below in relation to FIG. 4.

FIGS. 2A-2F illustrate an embodiment of a frame 220 of a macroencapsulation device. The frame may be a perimeter frame that suspends the membranes within an opening of the frame. The frame may extend around at least a portion, and in some embodiments around an entire, perimeter of the bonded membranes. The size and shape of the frame may be selected to maintain a maximum transverse dimension of the membranes at a smaller second maximum transverse (e.g., a width) dimension after mounting relative to a larger first maximum transverse dimension of the membranes in a flat configuration prior to mounting. The maximum transverse dimension may be measured in a plane in which the planar frame extends. For example, the maximum transverse dimension in the depicted embodiment may correspond to a diameter of the circular frame placed onto the bonded membranes. However, embodiments in which frames and membranes with different shapes and sizes are used are also contemplated.

As shown in the figures, the frame 220 may be circular in shape, although it should be noted that the frame may include any shape that corresponds to the shape of membranes to be attached thereto. The frame 220 may include an outer portion 222 and an inner portion 224. As shown in FIGS. 2E-2F, the outer portion 222 may have a rounded shape that tapers inward toward the inner portion 224, forming an inner perimeter surface 226 that extends around the frame 220.

The inner perimeter surface 226, or other portion of the frame configured to receive the one or more membranes disposed thereon, may include one or more reservoirs 228 corresponding to through holes, cavities, or other structures configured to receive a liquid adhesive there in during a mounting process. For example, the reservoirs may be arranged around the inner perimeter of the frame for mounting a perimeter of the membranes to the frame, as described in further detail below with respect to FIG. 4. The reservoirs 228 may be equally spaced around the inner perimeter surface 226, although the disclosure is not so limiting, and the reservoirs may be placed in any arrangement around the inner perimeter surface. The membranes may have holes or other markings that align with reservoirs 228 to position the membranes on the frame during a mounting procedure.

As shown in FIG. 2F, in some embodiments, the reservoirs may be tapered holes that extend through the frame from a first side to a second side of the frame opposite the first side. The reservoirs 228 may be tapered such that a diameter D1 of the reservoir on a first side is less than a diameter of the reservoir on a second side opposite the first. In some embodiments, the reservoirs 228 may not extend entirely through the frame such that the reservoir may only have an opening on the first or second side of the frame.

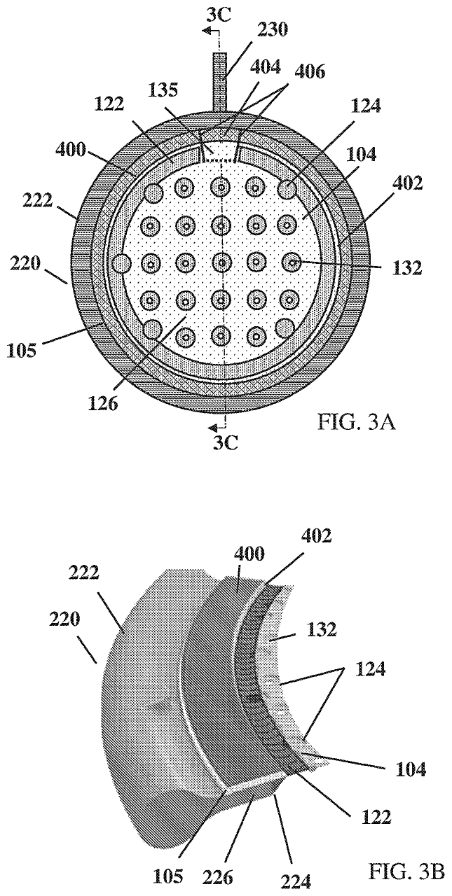

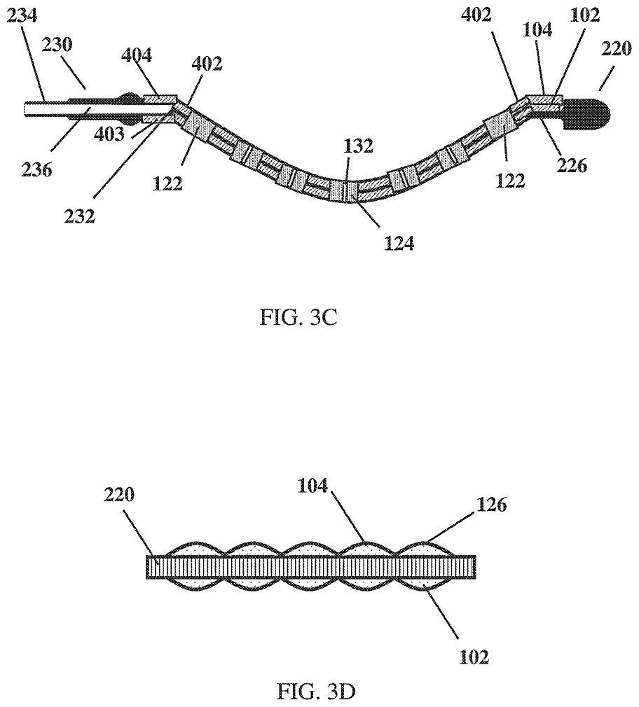

Going back to FIGS. 2A-2D, the frame 220 may include a fill port 230 that extends from an outer portion 222 to an inner portion 224 of the frame. The fill port 230 may include an opening 232. The opening 232 may be flush with an adjacent surrounding inner portion 224 of the frame such that the opening does not project into the inner portion 224 of the frame. A thickness of the inner perimeter surface 226 may increase surrounding the opening to accommodate a channel 236 (sec FIG. 3C) that extends from the opening through the fill port. The fill port 230 may also include a projection 234 that extends outwards from the outer portion of the frame. The channel 236 may extend from the opening 232 through the projection 234 such that when a membrane is attached to the frame, a desired material may flow through the fill port into an internal volume of the membrane.

FIGS. 3A-3D depict one embodiment of a macroencapsulation device after the membranes have been mounted to a corresponding frame. FIG. 3A is a front view of the device and FIG. 3B is a perspective cross sectional view of a portion of the frame-membrane interface. As shown, a bonded membrane including a first and second membrane (only the top surface of the second membrane 104 is shown in FIGS. 3A-3B) is connected to an inner perimeter surface 226 of a frame 220. The frame 200 extends around an entire perimeter of the bonded membranes, but the disclosure is not so limited and the frame may extend around a portion of the bonded membranes in some embodiments. The size and shape of the frame may be selected to maintain a maximum transverse dimension of the membranes at a smaller second maximum transverse dimension after mounting where the maximum transverse dimension may be measured in a plane in which the planar frame extends. For example, the maximum transverse dimension in the depicted embodiment may correspond to a diameter of the circular frame placed onto the bonded membranes. However, embodiments in which frames and membranes with different shapes and sizes are used are also contemplated. Without wishing to be bound by theory, the ratio of the first larger maximum transverse dimension prior to mounting and the second smaller maximum transverse dimension of the bonded membranes may control the volume of the internal volume disposed between the membranes once the membranes are filed with a therapeutic composition such as a population of cells.

As described above with respect to FIGS. 1A-1B, the membranes used to form a macroencapsulation device may include a bonded perimeter 122 that forms a seal extending around an internal volume disposed between the first and second membranes, which again may correspond to a single folded over membrane or two separate membranes. In the depicted embodiment, the bonded membrane is disposed on the frame 220 such that it covers the inner perimeter surface 226 of the frame with an outer perimeter 105 of the bonded membrane positioned at or near the outer edge of the inner perimeter surface 226, exposing only the exterior portion 222 of the frame. An adhesive layer 400 attaches the bonded membrane to the inner perimeter surface 226, or other appropriate portion, of the frame. The adhesive layer 400 may extend around the entire inner perimeter surface of the frame, though embodiments in which other types of connections, e.g. welding, are used or the membranes and frames are only bonded to one another along a portion of the frame or membrane perimeter are also contemplated. Appropriate adhesives used may also include UV or heat cured biocompatible adhesives including, but not limited to, urethanes, epoxies, or acrylates. An appropriate adhesive used may include epoxy-acrylate copolymers such as Epotek and/or Cyberlite. Alternatively, appropriate adhesives may include, but not be limited to molten thermoplastics in heat staking or welding applications, such as polycarbonates, polypropylenes, polyethylenes, ethylene vinyl acetate, polyether (ether ketone), polyvinyl chloride (PVC), polyvinylidene fluoride (PVDF), polystyrene, acrylonitrile butanedione styrene (ABS), polyurethane, and/or polymethyl methacrylate (PMMA).

As shown, the bonded perimeter 122 of the membranes is located radially inward from the outer perimeter 105 of the bonded membrane. An unbonded portion of the bonded membrane or buffer region 402 separates the bonded perimeter 122 and the adhesive layer 400 that bonds the membrane to the inner perimeter surface 226 of the frame. In some embodiments, a transverse dimension (e.g., a width) of the buffer region 402 between the frame and bonded portion of a membrane may be greater than or equal to 350 μm, 400 μm, 500 μm, 750 μm, and/or 1 mm. In some embodiments, the transverse dimension (e.g., a width) of the buffer region 402 may be less than or equal to 2 mm, 1.5 mm, 1.25 mm, 1 mm, 750 μm, and/or 500 μm. Combinations of the foregoing ranges are contemplated including, for example, a transverse dimension (e.g., a width) of the buffer region may be between or equal to approximately 350 μm and 750 μm, 400 μm and 750 μm, 350 μm and 2 mm, or other appropriate combinations of the foregoing. However, embodiments, with a buffer region with a transverse dimension between the adhesive layer 420 and the bonded perimeter 122 that is different from those noted above are also envisioned. A liquid adhesive used to bond the membrane to the frame may have certain viscosity and wicking characteristics that are balanced with the properties of the membranes (e.g. porosity, tortuosity, etc.) to prevent the liquid adhesive from entering the buffer region 402 when bonding the membranes to the frame. The adhesive may also have an elastic modulus greater than an elastic modulus of the flexible membrane and less than an elastic modulus of the rigid frame in some embodiments. As such, the elastic modulus of the macroencapsulation device may decrease from the outer frame to the adhesive layer to the membrane, and the device may become more flexible proceeding from an exterior portion of the device toward the center of the device.

As a result of the above construction, the buffer region 402 may reduce, or substantially eliminate, stress concentrations near the bonded perimeter 122. This may reduce the risk of fatigue failure of the membrane. As described above, holes 132 may not be formed in the bonded portions 124 located near the bonded perimeter to further reduce stress concentrations near the bonded perimeter.

FIG. 3C shows a side sectional view of the macroencapsulation device of FIG. 3A taken along line 3C after the membranes have been mounted to a corresponding frame and prior to being filled with a desired material such as a population of cells. As shown, the device may include a first membrane 102, a second membrane 104, and a frame 220 that extends along at least a portion of the perimeter of the first and second membranes. The device is illustrated in an unfilled relaxed state where the extra surface area of the first and second membranes relative to the transverse cross-sectional area of the frame within which the membranes are mounted causes the bonded membranes to hang below the frame relative to a direction of gravity due to the resulting slack in the membranes.

The membranes 102 and 104 are bonded to the frame 220 on the inner perimeter surface 226, as shown on the right side of FIG. 3C. For a portion surrounding the fill port 230, however, as shown on the left side of FIG. 3C, the first membrane 102 is bonded to a first side of the frame and the second membrane 104 is bonded to a second side of the frame opposite the first side. As described in more detail below, flaps 403 and 404 are created in the first and second membranes by making cuts 406 in the membranes (see FIG. 3A) and sealing the flaps 403 and 404 around an opening 323 of the fill port 230. By sealing the flaps around the opening 323, the opening of the fill port is now in fluid communication with the internal volume disposed between the membranes.

Due to the bonded portions 124 located within an interior region of the device. through holes 132, and other appropriate features having already been formed on the membranes, the macroencapsulation device may now be easily filled with a desired material, such as a population of cells, with minimal additional processing and handling. The interior volume may be filled using a fill port 230, an opening in the perimeter bond, and/or any other appropriate method. In either case, after filling a macroencapsulation device with a desired material, the internal volume contained between the first and second membranes 102 and 104 may expand which may take up the slack in the membranes as the membranes are placed under tension in the filled configuration due to the internal volume between the membranes expanding. This may result in the first and second membranes being deformed such that the membranes generally extend in a direction that is approximately parallel to a plane of the frame 220, see FIG. 3D. Correspondingly, the first and second membranes may now extend outwards from opposing surfaces of the frame by approximately equal distance due to this increase in the internal volume of the now filled device. In instances where portions 124 of the membranes have been bonded together at a location located radially inwards from the frame, the expanded structure may again form a plurality of interconnected channels 126.

A macroencapsulation device may be filled through the fill port 230. For example, a population of cells, or other desired material, may be flowed into an interior volume of the macroencapsulation device formed between the outer membranes of the device. This may be accomplished through the fill port 230 or use of a scalable or removable port extending into the interior volume. Alternatively, there may be an opening in the perimeter bond and/or frame of the macroencapsulation device that may be subsequently sealed. While any appropriate inlet to the interior volume may be used to flow material into the interior volume of the device, the flow of this material may be controlled in a number of different ways to provide the desired filling of the interior volume. For example, in one embodiment, a pressure applied to an interior volume of the macroencapsulation device may correspond to a desired amount of tension present in the membranes of the device in the filled configuration. Accordingly, filling of the device may continue until a predetermined pressure and/or membrane tension threshold has been reached. However, any appropriate method for controlling the amount of material flowed into the interior volume may also be used as the disclosure is not limited in this fashion. This may include, for example, control based on an absolute volume of material flowed into the interior volume, time duration for a given flow rate, and/or any other appropriate control method.

FIG. 4 depicts one embodiment of a process for mounting a frame to a bonded membrane. As shown in FIG. 4, a frame 220 is disposed on a support 200. Once the frame is secured to the support, bonded membranes 102 and 104 may be disposed on the frame 200. In some embodiments, the membranes may be “slack mounted” to the frame. For example, the support 200 may include a curved surface 206 to deform the first and second membrane 102 and 104 from a first maximum transverse dimension before mounting (e.g., where the membranes are in a relatively flat planar configuration) to a second maximum transverse dimension after (e.g., where the membrane are deformed to conform to a shape of an underlying support 200). This concept of controlling an amount of slack in a membrane during mounting with a frame may refer to the mounting of at least two or more layers of flexible membranes (e.g., a first membrane and a second membrane) under a controlled relaxed tension to form a device comprising internal compartments of a defined volume and/or height when filled. In some embodiments, the curved surface of the support is a spherical dome as illustrated in FIG. 4. However, embodiments in which a support with a different shape is used are also contemplated.

In some embodiments, the membranes 102. 104 may include holes or other markings (not shown) disposed around a perimeter of the membranes that correspond with locations of the reservoirs 228 of the frame (see also FIG. 2A-2D) to align the membranes on the frame. In some instances, it may be desirable to maintain an orientation and/or position of a stack of membranes on a support during mounting to a frame. Accordingly in some embodiments, and as shown in the FIG. 4, a vacuum may be applied to one or more non-diffusive portions of the first and second membranes to maintain the first and second membranes proximate the curved support. For example, the support 200 may include a vacuum chamber 210 that is connected to a vacuum source, not shown, to provide a negative pressure. The vacuum chamber may be fluidly connected to one or more vacuum holes 212 disposed on a surface of the support 200. While the vacuum holes may be located at any desired portion of the support's surface, in some embodiments, the vacuum holes may be located on portions of the support's surface where a corresponding non-diffusive portion of the bonded membranes may be located including, for example, the bonded perimeter 122 of the membranes, a portion of the membranes located radially outwards from the bonded perimeter, the bonded portions 124 of the membranes located within the bonded perimeter, and/or any other appropriate portion of the membranes. Other methods of maintaining a position and/or orientation of the membranes relative to the underlying support may be used including, but not limited to, mechanical fixation, clamping, temporary adhesives, and/or any other appropriate temporary fixation method.

After positioning the frame 220 and the first and second membranes 102 and 104 in a support, the frame and membranes may undergo a number of different processes including bonding in one or more locations. FIG. 4 illustrates a bonding process of the first and second membranes to the frame. In certain embodiments, an adhesive, heat staking, welding (thermal, ultrasonic, etc.), mechanical fixation, or another appropriate method may be used to bond the frame and membranes at a plurality of locations around a circumference of the frame. This bonding may either be done sequentially or simultaneously depending on the manufacturing process. For example, the frame and membranes may be bonded together at each of the locations where the frame includes a reservoir 228 disposed around a circumference of the inner perimeter surface 226 of the frame (sec FIGS. 2A-2D). In the depicted embodiment, a bonding tool 500 may be used to create adhesion points between the frame and a portion of the first and second membranes at the one or more desired locations. The bonding tool 500 may correspond to a combination of a port used to dispense a curable adhesive and a light source that may be used to cure the adhesive once positioned on the frame and membranes.

In one specific embodiment, a bonding tool 500 (e.g., a needle, syringe) may deliver a liquid adhesive to a reservoir 228 of the inner perimeter of the frame 220. The bonding tool 500 may extend through the membranes 102 and 104 (e.g., by piercing the membranes or extending through pre-arranged holes in the membrane) to deposit the liquid adhesive into the reservoir 228. The liquid adhesive may then wick through portions of the first and second membranes above the reservoir 228. Alternatively, the bonding tool 500 may apply the liquid adhesive on a top surface of first membrane and/or second membrane above the reservoir and the liquid adhesive may wick through the membranes toward the reservoir 228. In other embodiments, the first and second membranes 102 and 104 may be disposed on the support 200 and the frame then disposed on top of the second membrane. A bonding tool 500 may extend through the reservoir 228 to deposit a liquid adhesive to a back surface of the second membrane. The reservoirs may be tapered in an insertion direction to allow easy insertion of the bonding tool. The liquid adhesive may wick through the membranes to bond the membranes to the frame.

Once liquid adhesive is applied and has time to wick through the membranes, the adhesive may be cured with a light source. Once a bond is formed in a desired location, the bonding tool 500 may be moved to an adjacent reservoir 228 (sec FIGS. 2A-2D) until a sufficient number of bonds are formed around the perimeter of the device. As shown in the figures, the frame may include equally spaced reservoirs disposed around the perimeter of the frame. although the disclosure is not so limited, and nonequal spacing may be used. As mentioned above, the duration of bonding and viscosity of the adhesive may be selected to avoid excessive wicking of the adhesive into the undesirable portions of the membranes (e.g., diffusive portions and/or buffer region 402). In some embodiments, the viscosity of the adhesive may be greater than or equal to approximately 100 cP, 200 cP, and/or 300 cP. The viscosity may also be less than or equal to approximately 1000cP, 750cP, and/or 500 cP. Combinations of the foregoing are contemplated including, for example, the viscosity may be between or equal to 100 cP and 1000 cP, or more preferably between 100 cP and 500 cP. Other viscosities both greater than and less than those noted above are also envisioned. The duration of bonding of the adhesive may be greater than or equal to approximately 5 seconds 10 seconds, and/or 15 seconds. The duration of bonding may also be less than or equal to approximately 60 seconds, 30 seconds and/or 20 seconds. Combinations of the foregoing durations are contemplated including, for example, a duration of bonding that is between or equal to 5 seconds and 60 seconds, or more preferably between 10 seconds and 30 seconds. Other durations both greater than and less than those noted above are also contemplated. Additionally, while a particular bonding method has been described other appropriate types of bonding may be used as noted above.

While the use of a liquid adhesive is described above, other appropriate types of bonding techniques such as heat staking, ultrasonic welding, laser welding, or any other appropriate bonding technique may also be used as the disclosure is not so limited.

The bonding locations may be predetermined such that an associated processor (not shown) may be configured to control the bonding tool 500 such that it is properly positioned relative to each reservoir 228 of the frame during the separate bonding procedures to form the bonds around the device perimeter. In some embodiments, the bonding tool 500 may include one or more sensors to detect the reservoir sites, such as through visual tracking, magnetic sensing, or other appropriate robotic system targeting methods. Thus the bonding tool and or support may include one or more sensors 110 distributed around a surface of the lower portion of the fixture 106 may communicate signals to the processor to implement feedback control of the bonding process.

After this initial fixation of the membranes to the frame, additional processing of the mounted frame and membranes may then be done including, for example, placing additional adhesive between the mounted frame and membranes to improve a bond there between. If the membrane was mounted on top of the frame in the initial fixation step (as shown in FIG. 4) then the device may stay fixed in support 200. If the frame was mounted on top of the membrane (with bonding tool extending through the reservoir to mount a back surface of the second membrane to the frame, as mentioned above), the macroencapsulation device including the frame and mounted membranes may be removed from the curved support and resecured in the support in an inverted position (i.e., with the membrane on top of the frame). The device may undergo another processing step that applies an adhesive layer 400 (see FIGS. 3A-3B) to further bond the membrane to the inner perimeter surface of the frame. A bonding tool may apply adhesive in sections between each reservoir 228, applying and curing adhesive in each section before moving onto an adjacent section, until the entire perimeter is bonded. Any wrinkles or creases in the membrane may be smoothed during the bonding process as well. As mentioned above, the duration of bonding and viscosity of the adhesive may be selected to avoid excessive wicking of the adhesive into the undesirable portions of the membranes (e.g., diffusive portions and/or buffer region 402) such that the seal and buffer region of the bonded membranes are disposed radially inwards relative to both the frame and the adhesive, or other form of connection, bonding the membranes to the frame.

After the frame is bonded to the membranes, the device may be removed from the support. To fill the interior volume of the device (e.g., with cells), portions of the first and second membranes surrounding the fill port 230 may be positioned on opposite sides of the frame and scaled around the opening 323 of the fill port 320. Referring back to FIG. 3A, starting on a first side of the frame with the membranes positioned on top of the frame, portions of the first and second membrane over the fill port area may be separated. In embodiments with an unfused portion 135 of the perimeter bond 122, the membranes may only need to be separated to up to the buffer region 402; however, in embodiments without an unfused portion 135 (i.e., the perimeter bond extends around the entire perimeter of the membrane), then the membranes may need to be separated past the perimeter bond. After the membranes are sufficiently separated, cuts 406 may be created in the first and second membranes on each side of the fill port 230. The cuts 406 may be perpendicular to the adhesive seal 400 and extend from the membrane perimeter 105 to the perimeter bond 122. The cuts 406 create a first flap 403 in the first membrane and a second flap 404 in the second membrane 104 opposite from the first flap. The second flap 404 is then folded back onto the top surface of the second membrane 104 to reveal the first flap 403 (see dotted fold line between cuts 406 in FIG. 3A). The first flap 403 of the first membrane is then pushed inward, passing the interior portion 224 of the frame such that it protrudes from a second side of the frame opposite the first side. The first flap 403 is then pulled onto the inner perimeter surface 226 on the second side of the frame, smoothing the flap to the make the flap rest flush on a first upwards oriented surface of the frame. The second flap 404 is pulled over the first side of the frame and smoothed to the make the flap rest flush on a second downwards oriented surface of the frame opposite from the first surface. An adhesive is then applied to the flaps and cured to seal the flaps around the fill port opening, though other bonding and sealing methods may also be used.

In the above embodiments, a frame is connected to an exterior surface of a first membrane 102 opposite from a second membrane 104. However, embodiments in which a frame 220 is disposed between the first membrane 102 and second membrane 104 are also contemplated. In such an embodiment, portions of the first and second membranes extending radially outward from a bond 122 extending along a perimeter of the membranes may be opened and the frame may be positioned between the membranes at a location disposed radially outward from the perimeter bond of the membranes. The first and second membranes may then be bonded to the frame using any appropriate bonding method as described previously. While a particular angular orientation of the frame, membranes, and underlying support has been depicted in the figures, it should be understood that any appropriate orientation of these components may be used as the disclosure is not limited in this fashion. In either case, the frame may still function to maintain a desired transverse dimension of the membranes once removed from the underlying support.

### Example: In Vivo Fatigue Testing

A Göttingen minipigs was used to study the mechanics of a macroencapsulation device. Designs of macroencapsulation devices that were tested included designs as described above as well as prior designs. The prior designs included bonded membranes mounted to a perimeter frame such that a seal perimeter of the membranes was disposed on the frame (i.e., there was no gap between an inner perimeter of the frame and the seal perimeter). Testing results of the prior designs, showing fatigue failure at the frame interface due to stress concentrations at these areas, motivated new designs of the macroencapsulation devices with stress relief zones at the frame interface.

In comparison to the perimeter frame, which has been tuned to allow only a small amount of flexibility, the region of the device comprised primarily of membrane is significantly more flexible, leading to a mechanical transition zone between the frame and membrane that is coupled with adhesive. In silico and nonclinical studies of prototype devices identified this area as the most likely point of fatigue failure in the device, which was revised in a subsequent version by adding a stress relief zone as described herein to strengthen the interface. To interrogate the mechanical durability of the device at the frame-membrane interface, a fatigue test was developed to accelerate the functional duration of testing to time points beyond the duration of the proposed nonclinical studies.

### Example: Ex Vivo Fatigue Testing

Ex vivo fatigue testing was done to simulate forces exerted by myofibroblast-anchored abdominal contraction within the center mesh of a macroencapsulation device by developing a biphasic, fully reversed loading strategy wherein a clamped membrane is cycled symmetrically through displacement extremes. During testing, the device frame was secured between two parallel aluminum plates as the center-mesh clamp was actuated in the axial direction, applying cyclic tension to the mesh defined by a relevant physiological load or, in the case of design exploration, an elevated load to enable rapid iterative feedback.

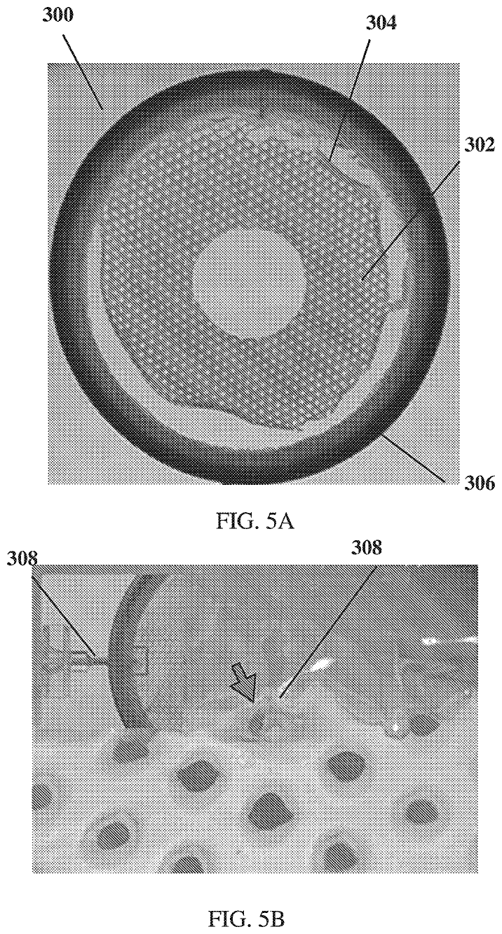

FIGS. 5A-5B depicts an embodiment of a prior design of a macroencapsulation device 300 after fatigue testing. In such an embodiment, the membrane 302 was mounted to the frame 306 such that the seal perimeter 304 was arranged at the frame interface. The device 300 also includes a fill port 308 that extends from the frame perimeter into the membranes. As shown in FIG. 5A, the membrane 302 has ruptured from the frame 306 at the seal perimeter 304 due to stress concentrations at the interface. Fatigue testing on prior designs and new designs showed that the prior design failed after approximately 10000 cycles, whereas the new designs failed after more than 30000 cycles. FIG. 5B shows the fill port interface and an enlarged view of a tear at the fill port interface caused by high stress concentrations. Internal fill port damage was observed in 52% of devices. New designs without internal fill ports resulted in improved fatigue life as elaborated on below.

### Example: Failure Mode Investigation

Tests were conducted to investigate potential causes of membrane failure. Results showed that failure of the devices was caused primarily by adhesive irregularity and lack of concentricity

### A. Manufacturability: Adhesive Application

One cause of device failure at the frame interface may be due to poor application of the adhesive by trainees. Devices made by experts may have subtle irregularities, while devices made by trainees may have gross irregularities. Test results confirmed that devices having gross irregularities failed early, with less than 1000 cycled to failure, compared with approximately 6000 cycles to failure with devices having subtle irregularities. Fatigue testing of the devices also showed failure of the membranes that matched failure of the devices tested in vivo. Prior designs required tight mounting tolerances and precise adhesive application to reduce risk of failure, which is difficult to automate and requires manufacturing my highly skilled individuals. The updated designs with stress relief zones and manufacturing methods as described above provide higher tolerances for adhesive application, which allows for manufacturing by lower skill level trainees or automation (e.g., using reservoirs around perimeter of frame as described above). For example, the addition of the reservoirs around the frame perimeter provide a reliable method to reduce the number of gross incursions which greatly affect the number of cycles to crack formation on the devices. Devices with gross incursions formed cracks at approximately 1000 cycles whereas devices with no incursions formed cracks at more than 7000 cycles. The fatigue testing methodology was developed specifically to mimic the forces exerted by myofibroblast-anchored abdominal contraction within the center mesh by developing a biphasic, fully reversed loading strategy wherein a clamped membrane was cycled symmetrically through displacement extremes. During testing, the device frame was secured between two parallel aluminum plates and a central portion of the membrane was clamped to a loading system, such as an Instron fatigue test fixture, to apply axial displacements to the membranes relative to the frame to apply cyclic tension to the membranes defined by a relevant physiological load or, in the case of design exploration, an elevated load to enable rapid iterative feedback. The cyclic loading forces applied during these fatigue tests varied between 30N and 45N depending on the specific fixture and frame being tested. By providing the reservoirs, the number of gross incursions in the new devices was approximately 5 whereas the prior devices had almost 20 gross incursions.

### B. Adhesive Quality

Tests were conducted to determine whether the quality of adhesive used may affect the frame interface. For example, it was investigated whether the adhesive degrading or changing properties affects the interface and if there were advantages to using an alternative flexible adhesive (e.g., Cyberlite). In the first phase of testing, dogbone shapes of the regular adhesive (Epotek OG198-54) and Cyberlite were tensile tested at 0, 3, 6, 9 and 12 months to determine the tensile strength at fracture. Results showed that Epotek was stable with no apparent embrittlement over time. For example, the adhesive had consistent tensile strength at break of approximately 20-25 MPa when tested at each time interval. The Epotek also showed a stable Young's modulus of approximately 1000 to 1250 MPa over 12 months. The Cyberlite, on the other hand, showed weakening over time, with a tensile strength of approximately 10 MPa at 0 months and less than 5 MPa at 12 months. The Yong's modulus of Cyberlite also decreased from approximately 500 MPa to approximately 100 MPa over 12 months.

Second phase of testing including fatigue testing the new designs of macroencapsulation devices (e.g., with stress relief zone) with different adhesive combinations: full Epotek, Cyberlute/Epotek combination, and full Cyberlite. Results showed that devices with full Eportek had over 10⁵ cycles to failure, Cyberlite/Epotek combination had 10+cycles to failure, and full Cyberlite had the lowest with less than 10+cycles to failure.

Results showed that relocation of the seal perimeter improves manufacturability and fatigue resistance of the device. For example, the new design reduces dependence on an operator by increasing tolerances, improves concentricity and adhesion uniformity, and reduces the complexity of visual inspection. Based on the presented testing of the current devices, the expected fatigue life of the device is approximately five years of continuous coughing, estimated to represent a peak load on the device at 11.6 N for 87,600 cycles.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A macroencapsulation device for housing a population of cells comprising:
a first membrane;
a second membrane disposed on the first membrane, wherein the first membrane and the second membrane are bonded together to form a seal extending around an internal volume disposed between the first membrane and the second membrane, wherein the seal is disposed radially inward from an outer perimeter of the first and second membranes, wherein the first membrane and/or the second membrane is semipermeable; and
a frame, wherein the first membrane and the second membrane are disposed on the frame, wherein the frame extends along at least a portion of the outer perimeter of the first and second membranes, and wherein the seal is disposed radially inwards from the frame.

2. The macroencapsulation device of claim 1, further comprising a buffer region disposed between the seal and the frame.

3. The macroencapsulation device of claim 2, wherein the buffer region creates a gap between the seal and the frame greater than or equal to approximately 400 microns and less than or equal to approximately 2 mm.

4. The macroencapsulation device of claim 3, wherein the gap is less than or equal to approximately 750 microns.

5. The macroencapsulation device of claim 3, wherein the gap is greater than or equal to 750 microns.

6. The macroencapsulation device of claim 1, wherein a Young's modulus of the frame is greater than a Young'smodulus of the first membrane and of the second membrane.

7. The macroencapsulation device of claim 1, further comprising an adhesive bonding the first membrane and the second membrane to the frame, wherein a Young's modulus of the adhesive is greater than a Young's modulus of the first membrane and of the second membrane and less than a Young's modulus of the frame.

8. The macroencapsulation device of claim 1, further comprising a plurality of reservoirs disposed around an inner perimeter of the frame.

9. The macroencapsulation device of claim 1, wherein the frame extends around the perimeter of the first and second membranes.

10. The macroencapsulation device of claim 9, wherein the frame extends completely around the perimeter of the first and second membranes.

11. The macroencapsulation device of claim 1, wherein the frame comprises a fill port including a channel that extends through the frame and includes an opening disposed on an inner perimeter of the frame in fluid communication with the internal volume.

12. The macroencapsulation device of claim 11, wherein the opening is flush with the inner perimeter of the frame.

13. The macroencapsulation device of claim 1, further comprising a plurality of bonded portions of the first and second membrane disposed radially inward from the frame that form a plurality of interconnected channels disposed between the first membrane and the second membrane.

14. The macroencapsulation device of claim 13, wherein at least some of the bonded portions include a through hole passing there through.

15. The macroencapsulation device of claim 14, wherein bonded portions that include the through holes are disposed radially inward from the seal extending around the internal volume.

16. The macroencapsulation device of claim 1, wherein at least one of the first or second membrane is comprised of ePTFE.

17. The macroencapsulation device of claim 1, wherein at least one of the first or second membrane is a semipermeable membrane.

18. The macroencapsulation device of claim 7, wherein the adhesive is an epoxy-acrylate copolymer.

19. The macroencapsulation device of claim 1, wherein the macroencapsulation device has a fatigue life of at least 70,000 cycles.

20. A macroencapsulation device for housing a population of cells comprising:
a first membrane;
a second membrane disposed on the first membrane, wherein the first membrane and the second membrane are bonded together to form a seal extending around an internal volume disposed between the first membrane and the second membrane, wherein the first membrane and/or the second membrane is semipermeable; and
a frame disposed on the first membrane or second membrane that extends along at least a portion of the perimeter of the first and second membranes, wherein the frame includes a fill port extending from an exterior portion of the frame to an interior portion of the frame, and wherein an opening of the fill port located on the interior portion of the frame is in fluid communication with the internal volume and is flush with an adjacent portion of the internal portion of the frame.

21. The macroencapsulation device of claim 20, wherein the fill port includes an opening at a first end disposed on the inner portion of the frame, and wherein a portion of the first membrane and a portion of the second membrane are sealed to opposite surfaces of the frame adjacent the opening of the fill port such that the opening of the fill port is disposed between the first membrane and the second membrane.

22. The macroencapsulation device of claim 21, wherein the fill port includes a projection extending from an exterior portion of the frame, and wherein the fill port includes a channel that extends through the projection to the opening.

23. The macroencapsulation device of claim 20, wherein the frame extends around the perimeter of the first and second membranes.

24. The macroencapsulation device of claim 23, wherein the frame extends completely around the perimeter of the first and second membranes.

25. The macroencapsulation device of claim 20, wherein the first and second membranes are configured to block passage of the population of cells out of the device.

26. The macroencapsulation device of claim 20, wherein an average pore size of pores in the first membrane and/or second membrane are between or equal to 1 nm and 2500 nm.

27. The macroencapsulation device of claim 20, wherein the average pore size of pores in the first membrane and/or second membrane are between 50 nm and 1200 nm.

28. The macroencapsulation device of claim 20, further comprising the population of cells disposed in the internal volume.

29. The macroencapsulation device of claim 28, wherein the population of cells include insulin secreting cells.

30. The macroencapsulation device of claim 20, wherein the internal volume includes a plurality of channels disposed between the first membrane and the second membrane.

31. The macroencapsulation device of claim 20, further comprising a plurality of bonded portions of the first and second membrane disposed radially inward from the frame that form the plurality of channels.

32. The macroencapsulation device of claim 20, wherein the seal is disposed radially inward from an outer perimeter of the first and second membranes.

33. The macroencapsulation device of claim 20, wherein the macroencapsulation device has a fatigue life of at least 70,000 cycles.

34. A method of forming a macroencapsulation device, the method comprising:
depositing a first membrane and a second membrane onto a frame, wherein the first membrane and the second membrane are bonded together to form a seal extending around an internal volume disposed between the first membrane and the second membrane, wherein the seal is disposed radially inward from an outer perimeter of the first and second membranes; and
connecting the frame to the second membrane and/or the first membrane along the outer perimeter of the first and second membranes at one or more locations located radially outward from the seal.

35-45. (canceled)

46. A method of forming a macroencapsulation device, the method comprising:
disposing a first membrane and a second membrane on a first surface of a frame, wherein the frame includes a fill port extending from an exterior portion of the frame to an interior portion of the frame, and wherein the frame includes a second surface located opposite from the first surface;
disposing a first flap of the first membrane on a portion of the first surface adjacent to the fill port;
disposing a second flap of the second membrane on a portion of the second surface adjacent to the fill port such that a portion of the fill port is disposed between the first flap and the second flap; and
sealing the first flap and the second flap with the frame such that the fill port is in fluid communication with an interior volume disposed between the first membrane and the second membrane.

47-56. (canceled)
